(19) United States
(12) Patent Application Publication
(10) Pub. No.: US 2023/0329738 A1
(43) Pub. Date: Oct. 19, 2023

(54) DERMABRASION DEVICE

(71) Applicants: UNIVERSIDAD DE LOS ANDES, Bogota (CO); FUNDACION SANTA FE DE BOGOTA (FSFB), Bogota (CO)

(72) Inventors: David BIGIO ROITMAN, Bogota (CO); Fernando Luis GARCIA AYOLA, Bogota (CO); Constanza MORENO SERRANO, Bogota (CO); Viviana GOMEZ ORTEGA, Bogota (CO)

(21) Appl. No.: 18/042,526

(22) PCT Filed: Aug. 30, 2021
(86) PCT No.: PCT/IB2021/057912

§ 371 Date: Feb. 22, 2023

(30) Foreign Application Priority Data
Aug. 31, 2020 (CO) NC2020/0010958

(51) Int. Cl. A61B 17/32

(52) U.S. Cl. CPC A61B 17/32; A61B 2017/320008; A61B 2017/00761

(57) ABSTRACT

A dermabrasion device having a surface (120) with protuberances (110) arranged in a pattern (160) defined by an abrasive group (130) with at least three protuberances (110), arranged in a first arc; and a second abrasive group (130) arranged in a second arc having a larger radius than the first arc and wherein the second arc is concentric with the first arc, each abrasive group (130) being formed by at least three protuberances (110) with different apex shapes. In addition, the abrasive group (130) has protuberances (110) that have different heights to remove unwanted tissue at different skin depths, preventing the creation of noticeable wrinkles or lines that may later cause scarring.

### FIELD OF THE DISCLOSURE

This disclosure relates to dermabrasion devices. Specifically, the present disclosure is related to devices for dermabrasion and manual debridement of cutaneous and subcutaneous tissue.

### DESCRIPTION OF THE PRIOR ART

Burns are injuries to the skin or deeper tissues caused by friction, sun, hot liquids, fire, electricity, or chemicals. Severity of most burns is related to the injury size and depth. Symptoms range from mild discomfort to a life-threatening emergency, depending on the burn size and depth.

Sunburns and minor scalds can be treated at home. Deeper or widespread burns and electrical burns need immediate medical attention, often in specialized burn units.

Patients with infections, devitalized tissue or deep burns on their skin are subjected to medical treatment because these affected tissues may compromise the function of connective, muscular, and nervous tissue, and even affect other internal organs. In addition, these affected tissues often leave severe scarring that leads the patient to suffer social rejection and emotional disorders. The treatment of the affected tissues is aimed at removing the affected tissue by means of tangential excision of the affected tissues and their immediate dressing. In order to remove the affected tissues, a process of dermabrasion or debridement and covering with biological or synthetic dressings is carried out until the epithelization of all the compromised areas is achieved. These procedures are painful, and the application of local anesthesia is necessary to reduce pain in patients.

Therefore, different dermabrasion devices have been developed, such as the one disclosed in document CN106108959A, which discloses a manual dermabrasion device, comprising a glove provided with removable abrasive sheets, where at least one abrasive sheet is provided on the thumb of the glove and the outer surface of said abrasive sheets is a rough grinding face. By using the glove, healthcare workers are allowed to experience the contact force with the patient's skin in real time and, thus, adjust the dynamics of the dermabrasion according to the wound surface condition. The CN106108959A dermabrasion device avoids excessive wound contact like dermabrasion devices known in the prior art, where the dermabrasion force is difficult to determine and easily causes excessive excision, sacrificing local function after surgery wound healing or sacrificing tissue condition of parabiosis due to secondary infection on the wound surface.

Additionally, document CN106108959A discloses that the grinding face of the abrasive sheet preferably corresponds to a medical sandpaper. Furthermore, preferably the grinding face of the abrasive sheet is provided with arc-shaped protuberances arranged in a matrix. In another embodiment, the protuberances are triangular and distributed in a matrix, where the installation direction of two adjacent rows of triangular protuberances are opposite.

On the other hand, document WO2003051284A2 discloses immunogenic compositions for the dermal delivery of an antigenic or immunogenic agent in combination with one or more excipients. The immunogenic compositions of the invention comprise an antigenic or immunogenic agent and at least one excipient that acts as an adjuvant, i.e., enhancing the immune response to the antigenic or immunogenic agent, once administered to the dermal compartment of the skin of a subject, e.g., intradermal or epidermal.

Furthermore, WO2003051284A2 discloses a micro-abrasion device including a substantially flat body or abrasive surface holder having a plurality of micro-protuberances extending from the lower surface of the holder. The holder is generally of sufficient thickness to allow the surface to join the micro-abrasion device base, thus allowing the device to be easily handled. Alternatively, a different handle or gripping device may be attached to or integral with the upper surface of the abrading surface support. The micro-protuberances are generally pyramidal in shape with sides extending to a point. The sides have a generally concave profile in cross-section display and form a curved surface extending from the abrasive surface holder to the point.

Document CN208355501U discloses a manual dermabrasion device. The device is fixed and attached by hand and its effect is reducing operator fatigue. In one embodiment the manual dermabrasion device comprises a thimble surrounding the fingertips with an outer dermabrasion layer, and an inner adhesive layer therein. In another embodiment the manual dermabrasion device comprises a plate covering the palmar side of a finger and a tape attached to said finger, where the plate has an outer dermabrasion layer.

Also, CN208355501U discloses that the outer surface of the dermabrasion layer is provided with a plurality of pyramidal protuberances, where the peak angle of the protuberance can be from 30° to 60°. Preferably, the peak angles of the conical protuberances comprise three specifications of 30°, 45° and 60°.

Finally, document WO2007015729A1 discloses an article useful for mechanical skin rejuvenation techniques that is suitable for transferring mechanical energy from a handheld device to the skin in contact with the article. The article may be characterized by its durable abrasiveness, compressibility, displacement, and/or surface roughness. The article may be formed of a fibrous structure having a first major surface associated with an adhesive system and a second major surface, generally opposite to the first major surface. The second major surface is arranged and configured to reversibly engage a fastener of a motion generating unit. The invention also relates to a coupling device for coupling a powered apparatus to a disposable skin contact element.

The documents described above disclose manual or assisted dermabrasion or debridement devices, in some cases corresponding to dermabrasion or debridement of wounds or devitalized tissue. The disclosed devices correspond to gloves, finger coverings, or machined items comprising an abrasive surface. Likewise, the abrasive surfaces comprise protuberances that can be pyramidal, conical with patterns that correspond to linear matrices. Also, some disclosures highlight the importance of performing the abrasive method manually, allowing operators who can adhere to or adapt an abrasive surface in their hands to remove skin from a patient by identifying the force that can be applied to patients to carry out dermabrasion without damaging the skin.

However, the linear arrays of the abrasive surfaces in the disclosed documents create grooves on the skin of patients, which would cause deep scarring in patients. On the other hand, mechanical dermabrasion and debridement devices make it difficult to control the amount of force medical personnel apply to patient's burns when removing devitalized tissue, so the applied force could be too much or too little. On the other hand, microdermabrasion devices are usually used in cosmetic procedures that would not remove all the devitalized tissue, which could leave tissue remains that would cause infections in the skin of patients.

Additionally, devices used for dermabrasion are mostly rustic devices such as hardware store sandpaper, which design is not focused on skin removal and from which particles that are embedded in cutaneous or subcutaneous tissues of patients can come off infecting or creating an immune system response to said particles. Also, devices such as dermatomes or skin slicers can be used, however, it is difficult to access areas of the skin with curved or cylindrical geometries or small areas with such devices.

### BRIEF DESCRIPTION OF THE DISCLOSURE

The device of the present disclosure is a dermabrasion device, comprising a surface with protuberances that are arranged in a pattern defined by an abrasive group with at least three protuberances, arranged in a first arc, and a second abrasive group arranged in a second arc with a greater radius than the first arc and where the second arc is concentric with the first arc.

The dermabrasion device of the present disclosure allows for treating skin injuries of different types, such as burns, especially second-degree burns, acne, skin defects caused by surgery or trauma, dryness, tattoos, potentially cancerous skin spots, among others.

When performing the dermabrasion process with the device, devitalized tissue is removed to reduce the risk of infection in patients and create the bleeding dew dew that promotes tissue regeneration, improves healing, and helps adherence of skin grafts on the treated surface.

Also, the dermabrasion device of the present disclosure allows for the removal of devitalized tissue by having a configuration that helps it to access difficult skin areas, achieving complete dermabrasion and debridement of the skin surface that is burned or affected by other injuries.

### BRIEF DESCRIPTION OF THE DRAWINGS

FIG. 1 corresponds to a top view of one embodiment of the protuberance pattern.

FIG. 2 corresponds to an isometric view of a series of abrasive groups and different types of protuberances.

FIG. 3 corresponds to an isometric view of a nine-protuberance abrasive group.

FIG. 4A corresponds to a view of the YZ plane that determines the dimensions of protuberances and the abrasive group when traversing the direction in the X-axis.

FIG. 4B corresponds to a view of the XZ plane that determines the dimensions of protuberances and the abrasive group when traversing the direction along the Y-axis.

FIG. 4C corresponds to a view of the YZ and XZ planes that determine the dimensions of protuberances and the abrasive group when traversing the X and Y directions, respectively.

FIG. 5A corresponds to an isometric view of the fastening element and the gripping means, showing the protuberances on one of the faces of the fastening element.

FIG. 5B corresponds to a rear view of the fastening element and the gripping means.

FIG. 6A corresponds to a front view of a glove as a fastening element and the pattern of protuberances on the palm of the glove.

FIG. 6B corresponds to a side view of a glove as a fastening element and some abrasive groups.

### DETAILED DESCRIPTION

In order to understand this invention, “devitalized tissue” shall be understood as a body tissue with little blood supply and lack of oxygen supply.

In order to understand the present disclosure, “dermabrasion” shall be understood as the skin exfoliation procedure, through which superficial layers of the skin are removed.

In order to understand this invention, “debridement” shall be understood as the procedure by which dead, damaged, infected or devitalized tissue is removed.

In order to understand this disclosure, “dermabrasion device” or “device” shall be understood as the device indicated in the brief description, detailed description, drawings, abstract, and claims that allows for carrying out dermabrasion and debridement procedures.

The dermabrasion device (100) of the present disclosure allows for treating skin injuries of different types, such as burns, especially second-degree burns, acne, skin defects caused by surgery or trauma, dryness, tattoos, potentially cancerous skin spots, among others.

When performing the dermabrasion process and debridement through the device (100), devitalized tissue is removed to reduce the risk of infection in patients and create the bleeding dew that promotes tissue regeneration, improves healing, and helps adherence of skin grafts on the treated surface.

Also, the dermabrasion device (100) of the present disclosure allows for the removal of devitalized tissue by having a configuration that helps it to access difficult skin areas, achieving a complete dermabrasion of the burned skin surface.

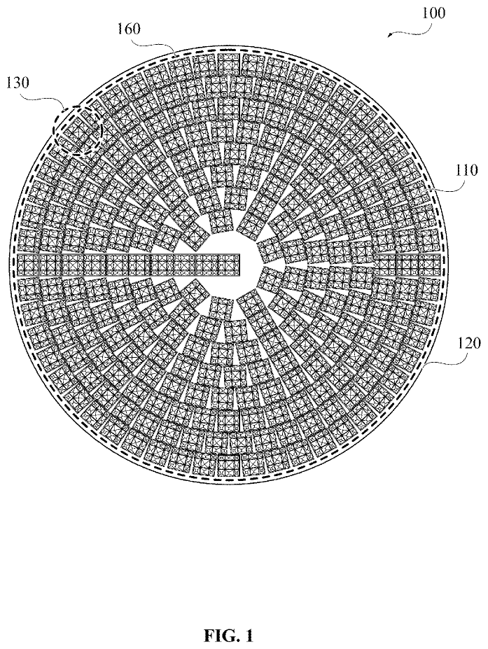

Therefore, and referring to FIG. 1, the present disclosure is a dermabrasion device (100), comprising a surface (120) with protuberances (110) that are arranged in a pattern (160) defined by an abrasive group (130) with at least three protuberances (110), arranged in a first arc; and a second abrasive group (130) arranged in a second arc having a greater radius than the first arc and where the second arc is concentric with the first arc.

Additionally, the device (100) for performing dermabrasion is placed on the burned skin of patients, wherein the abrasive groups (130) and the protuberances (110) are in contact with the skin to be removed. The healthcare professional presses on the skin to be removed by means of the device (100) and moves the device (100) tangentially to the patient's skin. As the device (100) is moved over the skin, the abrasive groups (130) remove devitalized tissue by means of the protuberances (110). In addition, in some embodiments of the present disclosure, the protuberances (110) and abrasive groups (130) are located on one of the surface faces (120) in such a way that rows of protuberances (110) are not formed to avoid creating grooves or dimples on the skin of patients.

The surface (120) is an extension of a sheet or object comprising two dimensions: length and width that are projected on the axes X, Y. On one of the surface faces (120) the protuberances (110) are arranged. Said protuberances (110) are elevations comprising three dimensions: length, width and height that are projected onto the axes X, Y, Z.

It will be understood in the present disclosure that pattern (160) is a succession of geometric figures, in this case of protuberances (110) that form abrasive groups (130), wherein the pattern (160) comprises a center on the surface (120) and abrasive groups around the center that follow each other forming arcs around the center.

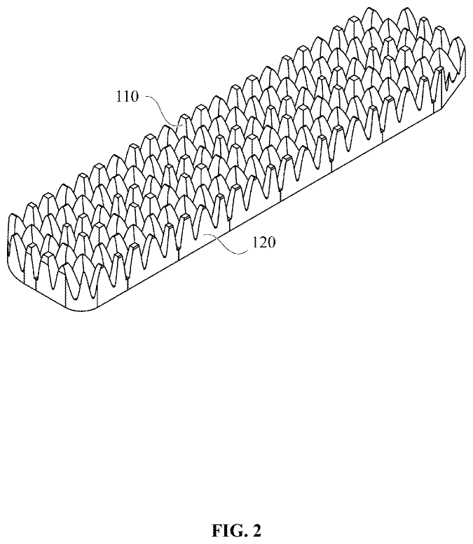

Referring to FIG. 2, the protuberances (110) can have geometry of pyramids, cones, cubes, prisms, orthohedrons, parallelepipeds, cylinders, and a base that can be in the form of squares, triangles, circles, rectangles, pentagons, trapezoids, ellipses, rhombuses, hexagon, heptagon, octagon, decagon, among other shapes. In addition, the protuberances (110) may have a peak which shape is selected from the group consisting of domed peak, truncated peak, and pointed peak.

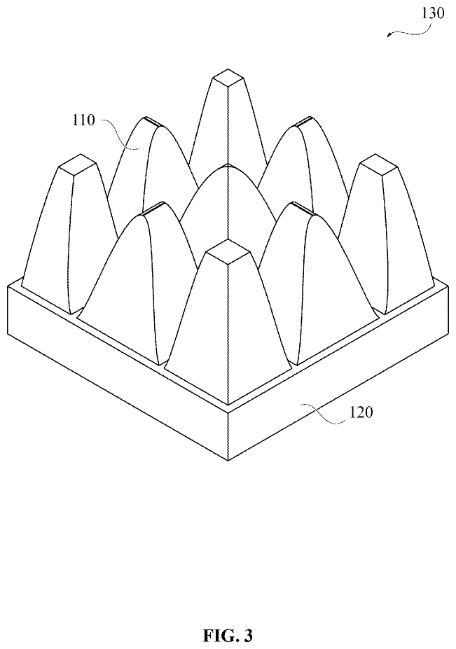

In one embodiment of the invention, the protuberances (110) are truncated at their peak or Z-axis, as illustrated in FIG. 3. Also, preferably, the protuberances (110) have pyramid or cone geometries. The protuberances (110) allow removal of unwanted epithelial tissue, such as devitalized tissue, when in contact with skin of patients. When performing tangential movements to the skin of a patient with the dermabrasion device (100), protuberances pull on segments of skin that come off the rest of the cutaneous or subcutaneous tissue due to the force applied.

On the other hand, the abrasive groups (130) are arrays of ordered protuberances (110), which in turn are arranged to form a pattern (160), as illustrated in FIG. 1 and FIG. 3. Said pattern (160) of the dermabrasion device allows for tissue removal in a homogeneous fashion.

Referring to FIG. 3, in one embodiment of the present disclosure each abrasive group (130) comprises at least three protuberances (110) with different peak shapes. Also, the abrasive group (130) comprises protuberances (110) having different heights to remove devitalized tissue at different levels of skin depth and avoid creating marked lines or grooves that produce scars later.

It is worth mentioning that for the removal of devitalized tissue, several factors must be considered, such as the adequate amount of devitalized tissue to be removed, homogeneous removal of said devitalized tissue without leaving grooves on the skin, and the required removal depth so bleeding dew takes place in the wound. The different heights of protuberances (110) in the present disclosure and different shapes of the peak allow removal of a sufficient amount of skin in a homogeneous way, without leaving grooves and helping medical personnel to control how much skin is removed.

Also, the abrasive groups (130) provide different levels of dermabrasiveness and debridement depending on the number of protuberances (110) that are part of the abrasive group (130), which also establishes the ideal location of each abrasive group on the surface (120). The level of dermabrasiveness and debridement of each abrasive group (130) will depend on the height, geometry, and type of peak of the protuberances (110), also the greater the radius of the arc on which the abrasive group (130) is arranged, the greater will be the number of protuberances (110) of the abrasive group (130).

In some embodiments of the invention, abrasive groups (130) have a minimum of three protuberances (110), preferably nine protuberances (110), and a maximum of twelve protuberances (110) per abrasive group (110).

As illustrated in FIG. 1, a first abrasive group (130) and a second abrasive group (130) may be provided on the surface (120) wherein the first abrasive group (130) is arranged around the center of the surface (120) where the center may have an abrasive group (130) or on the contrary be empty. The first abrasive group (130) is arranged in a first arc surrounding the center. On the other hand, the second abrasive group (130) is arranged in a second arc next to or behind the first arc of the first abrasive group (130).

In another embodiment of the invention, the second arc, where the second abrasive group (130) is located, comprises a greater radius than the first arc and where the second arc is concentric with the first arc, when the second arc is located behind the first arc.

In addition, in another embodiment of the invention, the second arc is located next to the first arc, where the second abrasive group (130) is in the second arc and the second arc has the same radius as the first arc, where the first and second arch are adjacent.

In one embodiment of the invention, the larger radius arcs have abrasive groups (130) with a different number of protuberances (110) than the smaller radius arcs. In another embodiment of the invention, the larger radius arcs have abrasive groups (130) with a greater number of protuberances (110) than the smaller radius arcs.

In one embodiment of the invention, on one of the surface faces (120) there are n abrasive groups (130) wherein the surface (120) is completely covered by the abrasive groups (110) as illustrated in FIG. 2.

On the other hand, as mentioned above, the pattern (160) comprises abrasive groups (130) wherein each abrasive group comprises protuberances (110). The abrasive groups (130) are arranged in a pattern (160), which can be radial or circular. This allows for homogeneity to be achieved when removing the skin with the device of the present disclosure. This pattern (160) and its configuration prevents grooves or inhomogeneous removal from being generated in the skin.

In some embodiments of the invention, the pattern (160) allows the protuberances (110) to be distributed in a way that they do not align with the protuberances (110) of other adjacent abrasive groups (130), this allows the health professional to use the device of this invention without generating grooves on the patient's skin.

Additionally, the choice of the number of protuberances (110) and their different shapes and heights provides different abrasiveness degrees of the dermabrasion device. The combination of shapes, heights, and types of peaks on the protuberances (110) and the configuration of the abrasive groups on the surface (120), allow for reaching an ideal abrasiveness according to the type of skin, wound, and location on the body of a patient.

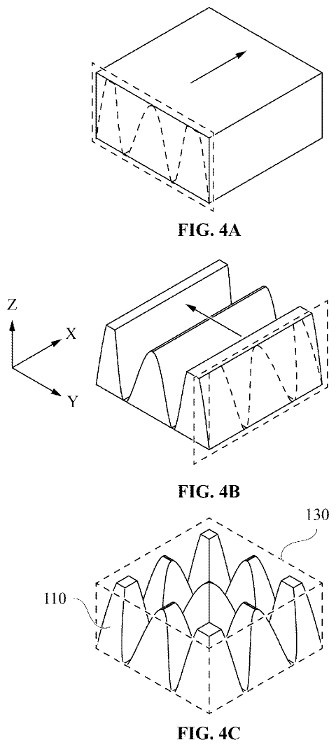

In one embodiment of the invention, the pattern (160) is mathematically defined as the volume (as illustrated in FIG. 4C) resulting from the intersection of the areas under the curve of the projection along the X-axis of the curve in the plane YZ (as illustrated in FIG. 4A) and of the projection along the Y-axis of the curve in the plane XZ (as illustrated in FIG. 4B) from the equation:

$$f ( h ) = \frac{a}{3} {sen}^{2} ( h ) + b * sen ( h ) cos ( h ) + \frac{c}{6} {cos}^{2} ( h ) sen ( h ) + \frac{d}{2} {cos}^{3} ( h )$$

where a, b, c, and d are real integers and h is the variable of the function.

In one embodiment of the invention, the variable values of the mentioned equation may be any number greater than 0. In a preferred embodiment of the invention, the variable values to determine the dimensions of the abrasive groups (130) are:
a=10;
b=2;
c=5; and
d=1.

These values define a pattern (160) that produces a homogeneous removal and a number of abrasive groups for the removal of devitalized tissue.

Referring to FIG. 4C, it is noticed that the abrasive group profile (130) is a sinusoidal profile which includes protuberances (110) of different heights, which allows for complete removal of a patient's devitalized tissues.

Therefore, in order to achieve abrasiveness, the abrasive groups (130) have dimensions on the X-axis between 20 mm and 50 mm, preferably 25 mm; on the Y-axis between 20 mm and 50 mm, preferably 25 mm. The choice of dimensions X and Y of the abrasive groups (130) are directly related to the level of abrasiveness required according to the condition of the patient's skin to be treated and the ability of the abrasive groups (130) to adapt to a specific surface.

In addition, the dimensions of the protuberances can be X-axis between 0.5 mm and 2 mm, preferably between 0.7 mm and 1.2 mm; Y-axis between 0.5 mm and 2 mm, preferably between 0.7 mm and 1.2 mm; Z-axis between 1 mm and 4 mm, preferably 1.524 mm. The choice of this shape and dimensions allows for achieving skin removal efficiency of each element.

On the other hand, on one of the surface faces (120) a fastening element (150) is arranged, where said fastening element (150) is selected from the group consisting of gloves, plates, cylinders or solid elements with their gripping means (140). The solid element can have a concave, convex, cylindrical, spherical, pyramidal, conical, or flat shape. On the other hand, the gripping means (140) can be selected from the group consisting of straps, handles, knobs or any element that allows a user to grip the dermabrasion device (100) in an ergonomic way.

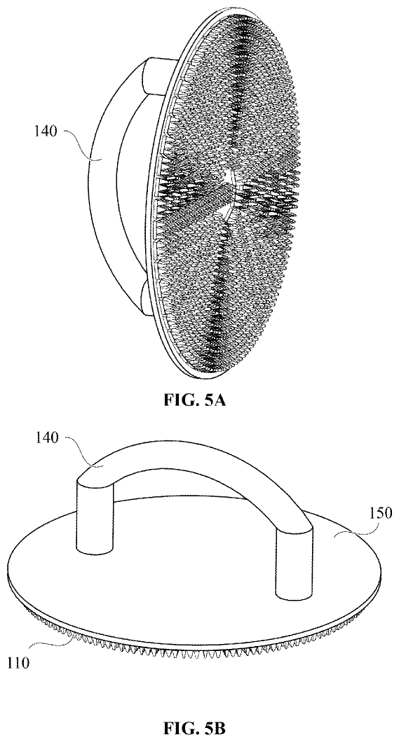

Referring to FIG. 5A and FIG. 5B the surface (120) is arranged on a solid disc-shaped element as a holding element (150) which comprises a handle as a gripping element (140) to be held by a user while performing the dermabrasion or debridement process.

In different embodiments of the invention, when the solid element has a flat shape in which the surface (120) is provided on one side and a gripping means is provided on the other side, the device is preferably used on skin surfaces relatively flat like the back and palms of the patient's hands and feet. Conversely, when the fastening element (150) is a glove or a cylindrical solid element, the dermabrasion device is preferably used on curved regions such as the neck, arms, between the fingers, ears, behind the ears, or legs.

In one embodiment of the invention, the surface (120) can be detached from the solid element to adhere to another solid element with a different geometry depending on the area of the patient's skin to be treated. For example, in a first abrasion process, the surface (120) is arranged with the abrasive groups (130) in a flat element, which is used to abrade areas of the back and soles of the hands and feet of a patient. In a second abrasion process, the surface (120) with the abrasive groups (130) detaches from the solid element in a flat shape and is arranged on a concave solid element to carry out the abrasion process on the patient's arms and legs.

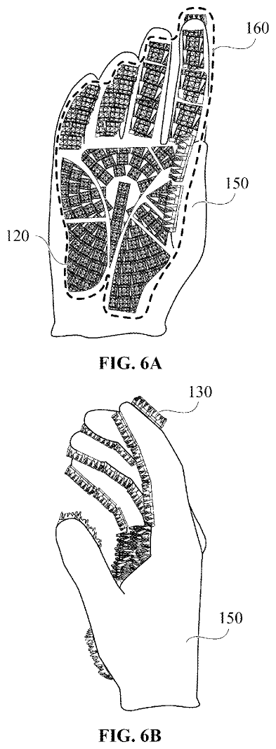

On the other hand and referring to FIG. 6A and FIG. 6B, if the gripping element (140) is a glove, the surface (120) can cover the entire palm of the glove. In this case, the surface (120) is divided into sections to allow the health professional to use the glove ergonomically, managing to extend and flex the fingers and generate the proper movements of the hand.

In particular, the sections of the surface (120) are divided according to the part of the palm of the hand overlapped by the glove. Therefore, the surface sections (120) are arranged over the distal phalanges, medial phalanges, proximal phalanges, hypothenar, thenar, distal palmar, and proximal palmar without obstructing the hand creases where the fingers and palm flex, as illustrated in FIG. 6.

The previous configuration allows for a greater contact area and allows the health professional to identify the amount of force to apply for carrying out the dermabrasion and/or debridement process. Also, the areas of the glove that are free of abrasive groups (130) facilitate the ergonomic movement of the hand. Additionally, and as shown in FIG. 6, the glove on the back of the index finger and thumb can have at least one abrasive group (110) to have a greater covering area and facilitate abrasion in areas of the patient's body with difficult or high complexity access such as the face or between the fingers.

In one embodiment of the invention, the fastening element (150) is made of a material selected from TPU, Latex, Aluminum, Medical Steel, Nitrile, PET, PLA, PETG, HDPE, among others. Said materials are biocompatible and sterile to avoid contaminating the tissue with which the dermabrasion device comes into contact.

Also, the fastening element (150) and the surface (120) can be joined by an adhesive selected from polyurethane adhesives, cyanoacrylates, moisture-curing silicones, modified silanes, acrylates, epoxy resins, among others.

In one embodiment of the invention, the surface (120) together with the protuberances (110) are made by a manufacturing process selected from among 3D printing, deposition, adhesion, extrusion, injection, and combinations thereof.

In one embodiment of the invention, the material of the protuberances (110) can be thermoplastic polyurethane (TPU), thermoplastic elastomer (TPE), high-density polyethylene (HDPE), polypropylene (PP), among other materials with similar properties and biocompatibility and sterilization features.

In one embodiment of the invention, the abrasive group (130) and protuberances (110) are made of a material having a Young's modulus between 0.01-5 GPa.

Also, in some embodiments of the disclosure the protuberances (110) and surface (120) are made of the same material that is selected from a biocompatible material. Biocompatible materials are deemed to be materials accepted for use in biological tissues, which preferably comply with the ISO-10993 standard: “Biological Evaluation of Medical Devices.” Some materials are for example: Polyaryl Etherketone (PAEK), Polyether Etherketone (PEEK), High Density Polyethylene (HDPE), Ultra High Molecular Weight Polyethylene (UHMWPE), Polymethyl Methacrylate (PMMA), Commercially Pure Titanium (ASTM F67), Titanium Alloys (ASTM B265), and AISI 316L stainless steel, collagen, silicone, glycolic acid, polyglycolic acid lactic acid, polydioxanone, polycaprolactone, chitosan, lactic-co-glycolic acid, polylactic-co-glycolic acid, co-polymers of the above and mixtures thereof.

In addition, depending on the choice of fastening element (150), the support surface (120) can vary its flexibility or elasticity features. If it is elastic like a glove, the surface (120) has a Young's modulus in the range of 10 MPa to 10,000 MPa. If the fastening element (150) is rigid, regardless of its shape, it is not necessary for the surface to have a Young's modulus as defined above.

Finally, the dermabrasion device of the present disclosure, in addition to removing devitalized tissue, can be used for exfoliation, aesthetic procedures, dead skin removal, skin cancer treatment, among others.

### Example 1

An abrasive group (130) defined by the intersection of the areas under the curve of:
The projection on the X-axis of the curve in the YZ plane of the equation:

General:

$$f ( x ) = \frac{a}{3} {sen}^{2} ( x ) + b * sen ( x ) cos ( x ) + \frac{c}{6} {cos}^{2} ( x ) sen ( x ) + \frac{d}{2} {cos}^{3} ( x )$$

Where a, b, c, and d are real integers and h is the variable of the function.

Preferably:

$$f ( x ) = \frac{10}{3} {sen}^{2} ( x ) + 2 sen ( x ) cos ( x ) + \frac{5}{6} {cos}^{2} ( x ) sen ( x ) + \frac{1}{2} {cos}^{3} ( x )$$

The projection on the Y-axis of the curve in the XZ plane of the equation:

General:

$$f ( y ) = \frac{a}{3} {sen}^{2} ( y ) + b * sen ( y ) cos ( y ) + \frac{c}{6} {cos}^{2} ( y ) sen ( y ) + \frac{d}{2} {cos}^{3} ( y )$$

Where a, b, c, and d are real integers and h is the variable of the function.

Preferably:

$$f ( y ) = \frac{10}{3} {sen}^{2} ( y ) + 2 sen ( y ) cos ( y ) + \frac{5}{6} {cos}^{2} ( y ) sen ( y ) + \frac{1}{2} {cos}^{3} ( y )$$

As a result, it was determined that the abrasive group (130) comprised 9 protuberances.

### Example 2

A surface (120) was designed with protuberances (110) arranged in a pattern (160) where the abrasive group (130) of Example 1 was used and arranged over a first arc surrounding the center of the surface (120). A second abrasive group, like the one in Example 1, was arranged on a second arc that had a greater radius than the first arc with respect to the center of the surface (120).

The first arc and the second arc were located in such a way that the protuberances (110) of different abrasive groups (130), observed from a top view, do not trace a straight line. This was repeated until the surface (120) was completely covered.

### Example 3

A dermabrasion device was designed where the surface (120) of Example 2 was arranged on a fastening element (150) like the one in FIG. 5A, wherein the fastening element (150) was rigid and completely flat. In addition, the fastening element (150) had a gripping element on one of its faces by means of which a health professional holds the dermabrasion device (100) while performing the dermabrasion and/or debridement procedure.

### Example 4

A dermabrasion device was also designed, where the surface (120) of Example 2 was used and divided into several sections, which were placed on a glove-shaped fastening element (150) like the one illustrated in FIGS. 6A and 6B.

1. A dermabrasion device, comprising:
a surface (120) with protuberances (110) that are arranged in a pattern (160) defined by:
an abrasive group (130) with at least three protuberances (110), arranged in a first arc; and
a second abrasive group (130) arranged in a second arc having a greater radius than the first arc and where the second arc is concentric with the first arc.

2. The dermabrasion device of claim 1, wherein the larger radius arches have abrasive groups (130) with a greater number of protuberances (110) than the smaller radius arches.

3. The dermabrasion device of claim 1, wherein the protuberances (110) have different heights.

4. The dermabrasion device of claim 1, wherein each protuberance (111) has a peak with a shape selected from the group consisting of domed peak, truncated peak, and pointed peak.

5. The dermabrasion device of claim 4, wherein the abrasive groups (130) have protuberances (110) with different peak shapes.

6. The dermabrasion device of claim 1, wherein the abrasive groups (130) comprise between three and twelve protuberances (110).

7. The dermabrasion device of claim 1, wherein the abrasive groups (130) are of different dimensions and number of protuberances (110).

8. The dermabrasion device of claim 1, wherein the pattern (160) is defined as the volume resulting from the intersection of the areas under the curve of the projection along the X-axis of the curve in the YZ plane and of the projection along the Y-axis of the curve on the XZ plane of the equation:
$$f ( h ) = \frac{a}{3} {sen}^{2} ( h ) + b * sen ( h ) cos ( h ) + \frac{c}{6} {cos}^{2} ( h ) sen ( h ) + \frac{d}{2} {cos}^{3} ( h )$$
where a, b, c, and d are real integers and h is the variable of the function.

9. The dermabrasion device of claim 8, wherein the values of the equation variables to determine the dimensions of the abrasive groups (130) are:
a=10;
b=2;
c=5; and
d=1.

10. The dermabrasion device of claim 1, wherein the surface (120) is divided into sections.

11. The dermabrasion device of claim 1, wherein the surface (120) together with the protuberances (110), is made by a manufacturing process selected from among 3D printing, deposition, adhesion, extrusion, injection, and combinations thereof.

12. The dermabrasion device of claim 1, wherein the dermabrasion device is arranged on a fastening element (150), where said fastening element (150) is selected from the group consisting of gloves and solid elements with a gripping means (140).

13. The dermabrasion device of claim 1, wherein the protuberances (110) have geometry of pyramids, cones, cubes, prisms, cuboids, parallelepipeds, cylinders, and a base that can be in the form of squares, triangles, circles, rectangles, pentagons, trapezoids, ellipses, rhombuses, hexagon, heptagon, octagon, or decagon.
